(19) United States
(12) Patent Application Publication
(10) Pub. No.: US 2012/0322106 A1
(43) Pub. Date: Dec. 20, 2012

(54) Production of Cellulases and Hemicellulases in Algal Biofuel Feedstocks

(76) Inventor: Bobban Subhadra, Albuquerque, NM (US)

(21) Appl. No.: 13/579,641

(22) PCT Filed: Feb. 18, 2011
(86) PCT No.: PCT/US11/25438

§ 371 Date: Aug. 17, 2012

Related U.S. Application Data
(60) Provisional application No. 61/338,389, filed on Feb. 18, 2010.

(51) Int. Cl. C12N 9/42; C10L 1/182; C07K 14/00; C12P 7/64; C12P 7/06; C12N 15/74; C10L 1/188; C12P 21/00

(52) U.S. Cl. 435/69.1; 44/385; 44/451; 435/134; 435/161; 435/471; 435/209; 530/350

(57) ABSTRACT

A method for producing hemicellulase and cellulase for use in processing cellulosic materials for biofuel production. The method involves transforming algal feedstock to express hemicellulase and cellulase and then collecting hemicellulase and cellulase that has been secreted into a culture medium by the algae. The method may further involve transforming the algal feedstock to express other recombinant products and/or processing the alga feedstock to produce biofuels.

### CROSS-REFERENCE TO RELATED APPLICATIONS

The following application claims benefit of U.S. Provisional Application No. 61/338,389, filed Feb. 18, 2010, which is hereby incorporated by reference in its entirety.

### BACKGROUND

There is a strong need for alternative energy options to meet growing energy consumption needs and depleting traditional energy sources. The recent increase in biofuel production from food crops has produced the unintended consequence of decreased food supply and increased food prices. Accordingly there is an increased demand for biofuels produced from non-food sources. Biodiesel made from fast-growing algae, enzyme hydrolysis of linocellulosic material sources such as forest waste, willow, and switch grasses, thermal depolymerisation of organic waste to form ‘biocrude’, and direct biological synthesis of more complex biofuels, each have potential. Of these many possible advanced feedstocks, algal biofuel and cellulosic bioethanol from switchgrass and other forest residues stand out as promising options because of the positive sustainability factors associated with them.

While the benefits of using non-food crops such as lignocellulosic materials to produce biofuels are clear, the costs and technical difficulties associated with the type of mass-scale production of lignocellulosic material-based biofuels are significant. See, e.g., Reinjnders L., Biomass and Bioenergy 34, 152-155 (2010), which is hereby incorporated by reference. Enzyme hydrolysis of lignocellulosic material relies on hemicellulases such as xylanase to break down the hemicelluloses surrounding the cellulose in the plant and cellulases such as endoglucanase, exoglucanase or cellobiohydrolase, and β-glucosidase, which are needed to break down cellulose into glucose. Accordingly, cost-effective mechanisms for high-yield production of cellulases and hemicellulases would go a long way in reducing costs and making commercial scale production of biofuels from lignocellulosic materials a reality.

Furthermore, due to the increased costs associated with alternative energy production, the ability to produce multiple types of biofuels and/or other valuable products in the same biorefinery using shared resources would be highly desirable.

### BRIEF DESCRIPTION OF THE DRAWINGS

FIG. 1 is a flowchart showing production of cellulase and hemicellulase using transformed algal strains according to an embodiment of the present disclosure.

FIG. 2 is a schematic illustration demonstrating transformation of algal strains to secrete cellulases and hemicellulases.

FIG. 3 is a flowchart of co-processing algal and cellulosic feedstocks for biofuel production according to an embodiment of the present disclosure.

FIG. 4 is a flowchart of processing recombinant proteins, cellulases and hemicellulases from algal biofuel biomass according to an embodiment of the present disclosure.

FIG. 5 is a schematic illustration of an exemplary biorefinery according to an embodiment of the present disclosure.

### BRIEF DESCRIPTION OF THE EMBODIMENTS

According to one embodiment, the present disclosure provides methods for inexpensive production of cellulases and hemicellulases which can be used to hydrolyze cellulose and hemicellulose. According to another embodiment, the present disclosure provides a method for co-producing both algae-based biofuels and lignocellulosic material biofuels. According to yet another embodiment, the disclosure provides a biorefinery capable of producing both algae-based and lignocellulosic material based biofuels.

### DETAILED DESCRIPTION

According to one embodiment, the present disclosure provides methods for inexpensive production of cellulases and hemicellulases which can be used to hydrolyze cellulose and hemicellulose. The mechanism by which cellulases and hemicellulases break down and hydrolyze lignocellulosic materials is described, for example, in Sticklan, M. B., Nature Reviews Genetics 9, 433-443 (June 2008), which is hereby incorporated by reference.

According to Wilson, D. B, Current Opinion in Biotechnology 20, 295-299 (2009) cellulases are currently the third largest industrial enzyme worldwide because of their use in cotton processing, paper recycling, as detergent enzymes, in juice extraction, and as animal feed additives. However, cellulases will become the largest volume industrial enzyme, if ethanol, butanol, or some other fermentation product of sugars, produced from biomass by enzymes, becomes a major transportation fuel.

Numerous studies have investigated the mechanisms of enzymatic hydrolysis of lignocellulosic substrates by cellulases produced by a variety of fungi. See for example, Rosgaard et al. Biotechnology Progress 22:2 493-498 (2006) which is hereby incorporated by reference. For example, it is known that T. reesei secretes high amounts of enzymes, up to 40 g·L⁻¹, that comprise a battery of endoglucanase, cellobiohydrolase, β-glucosidase, and different hemicellulytic activities that catalyze the degradation of the cellulose and hemicellulose of plant cell walls. Prior to enzymatic hydrolysis most lignocellulosic substrates undergo some sort of pretreatment to increase the accessibility of the substrate to enzymatic attack. The pretreatment should preferably result in removal of lignin and increase the available surface area and the substrate porosity and may also solubilize the hemicellulose. In particular, the removal of lignin has a large influence on the rate and extent of enzymatic hydrolysis of the substrate.

Generally, enzyme activity increases and often almost doubles for every 10° C. increase in temperature (up to a certain maximum temperature where inactivation occurs.) Accordingly, it may be desirable to conduct enzymatic hydrolysis at high temperature. Some filamentous fungi produce cellulases that retain relatively high cellulose-degrading activity at temperatures of 50-70° C., particularly species such as Thielavia terrestris, Thermoascus aurantiacus, Chaetomium thermophilum, Myceliophtora thermophila, and Corynascus thermophilus. Other fungi, for example from the Penicillium family such as P. funiculosum, display a broad profile of cellulytic enzymes, particularly with high-glucosidase activity compared to that of some T. reesei strains. See again, Rosgaard et al. Biotechnology Progress 22:2 493-498 (2006), incorporated above. Accordingly, it may be desirable to produce a similarly broad profile of cellulytic enzymes.

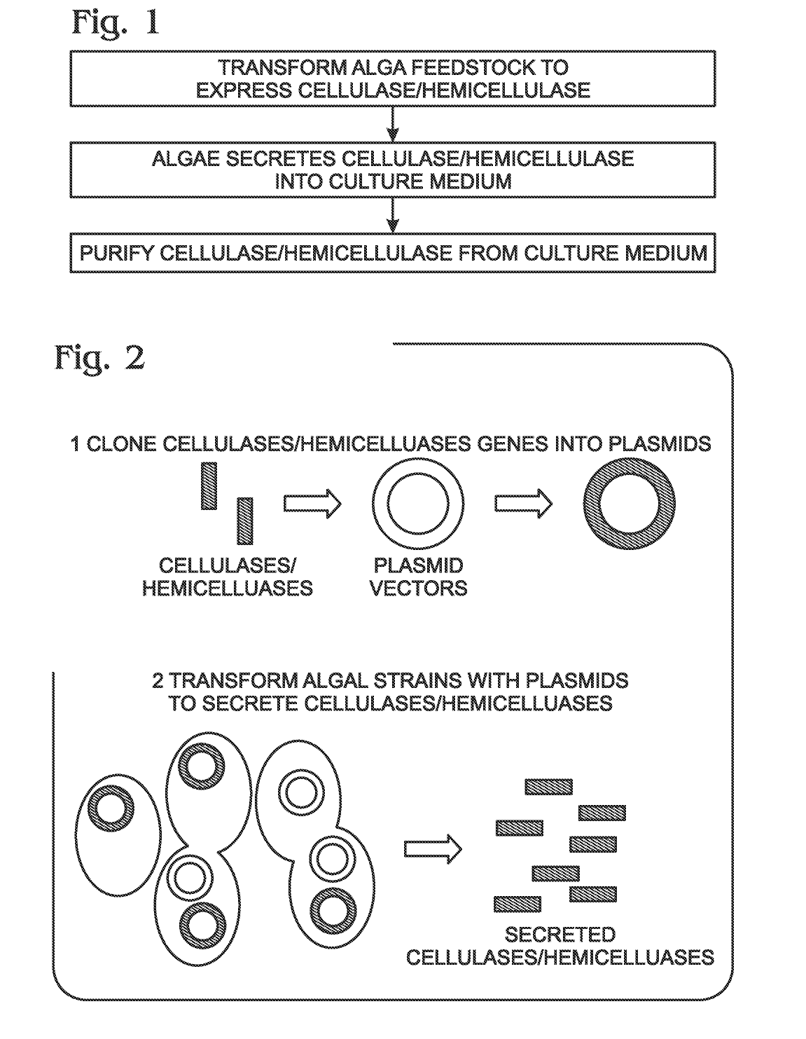

Turning now to FIG. 1, a first embodiment of the present disclosure is provided. As shown, algae feedstock is transformed to express cellulase and hemicellulase. The transformed algae then secretes cellulase and hemicellulase into the culture water, which can be collected and from which the enzymes can be purified.

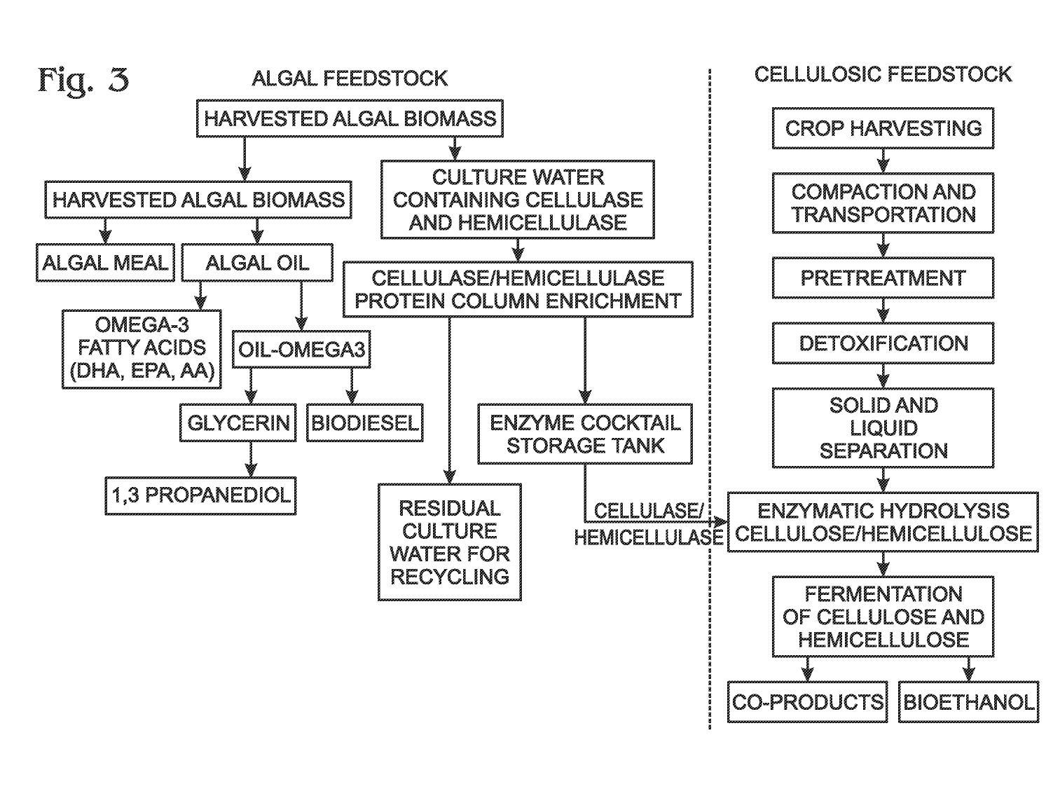

Turning to FIG. 2, a method of developing transgenic algal strains to secrete cellulases and hemicellulases is shown. As shown, cellulase and/or hemicellulase genes are cloned into plasmids which are then transformed into the algae. The transformed algal strains then secrete the cellulases and/or hemicellulases into the environment. According to an embodiment, known methods of transforming the algal strains may be used. For example, the green alga C. reinhardtii has long served as a model system for photosynthesis and flagellar function. This unicellular green alga will grow on a simple medium of inorganic salts in the light, using a photosynthesis system that is similar to that of higher plants to provide energy. C. reinhardtii will also grow in total darkness if an alternate carbon source, usually in the form of acetate, is provided. Both the 15.8 Kb mitochondrial genome (Genbank accession: NC001638 (See e.g., Vahrenholz C, Riemen G, Pratje E, Dujon B, Michaelis G. Curr Genet. 24 241-247 (1993)) and the complete >200 Kb chloroplast genome for this organism are available online (Genbank accession: BK000554 (Maul et al. 2002)). The current assembly of the nuclear genome is available online at http://genome.jgi-psf.org/Chlre3/Chlre3.info.html. The Chlamydomonas Center located at www.chlamy.org continues to be an informative resource to the Chlamydomonas community.

Over the last two decades, several highly efficient methods for nuclear, chloroplast and mitochondrial transformation have been developed for C. reinhardtii. Introduction of foreign DNA into the nuclear genome of C. reinhardii was initially performed using bombardment with DNA-coated microparticles, and/or agitation with glass beads or silicon carbide whiskers (See e.g., Debuchy, R. et al., EMBO Journal 8:2803-2809. (1989); Dunahay, T. G. 15 452-460 (1993); Gumpel and Purton Curr. Genet. 26:438-442 (1994), each of which is hereby incorporated by reference). Transformation efficiencies were found to be very low using all these methods. Molecular analysis of transformants revealed predominantly random recombination of transforming DNA into the nuclear genome, resulting in a distribution of expression levels caused by positional effects of the integrating DNA. Although the expression of re-introduced endogenous nuclear genes, particularly those that rescue nutritional auxotropes, has been relatively successful, the expression of heterologous genes transformed into the nucleus of C. reinhardtii remains problematic. More recently, electroporation was used to introduce foreign DNA as large as 14 kb into this organism (See e.g., Brown et al. Mol. Cell. Biol. 11:2328-2332 (1991)). Not only is this method quick and simple, it also yielded a transformation efficiency that was 2 orders of magnitude higher than previously described methods (See, e.g., Shimogawara et al. Genetics 148:1821-1828. (1998)). Currently, microprojectile particle bombardment appears to be the most efficient way of introducing DNA into the chloroplast genome of C. reinhardtii. Once inside the organelle, foreign DNA will usually integrate into the genome by homologous recombination (See, e.g., Boynton et al. Science 240, 1534-1538 (1988)). This highly reproducible protocol had allowed for studies involving specific gene disruptions and site directed mutagenesis on plastid genes. Even with these developments, the expression of recombinant therapeutic proteins, such as antibodies, in green algae is limited. It was not until 2003 when Mayfield et al. elegantly expressed human monoclonal antibodies in transgenic algal chloroplasts (Proc Natl Acad Sci USA 100:438-42. (2003)). In this work, C. reinhardtii chloroplast attpA or rbcL promoters were used to drive the expression of an engineered large single-chain antibody directed against herpes simplex virus (HSV) glycoprotein D. This antibody accumulated as a functional soluble protein in transgenic chloroplasts, and bound herpes virus proteins, as determined by ELISA assays.

This breakthrough serves as the first demonstration of microalgae as an expression platform for complex recombinant proteins, and is currently being utilized by Rincon Pharmaceuticals Inc, a San Diego-based biopharmaceuticals company for expression of monoclonal antibodies for use in cancer therapy. The bottleneck for genetic transformation of diatoms was resolved in 1995. Dunahay et al generated lines of transgenic Cyclotella cryptica and Navicula saprophila with plasmid vectors containing the E. coli neomycin phosphtransferase II gene using helium accelerated particle bombardment (See e.g., Dunahay et al. J. Phycol. 31:1004-12 (1995)). This was followed by the successful transformation of Phaeodactylum tricornutum (See, e.g., Apt et al. Mol Gen Genet 252: 572-579 (1996)) and Cylindrotheca fusiformis (See e.g., Fisher et al. J. Phycol. 35, 113-120 (1999)). A landmark transformation study was demonstrated by Zaslayskaia et al. in 2001 (Zaslayskaia et al., Science 292:2073-5 (2001)). Most diatoms are solely photosynthetic and lack the ability to grow in the absence of light. These investigators successfully engineered P. tricornutum, a photosynthetic diatom, to grow on exogenous glucose in the dark by transformation with the glucose transporter gene Glut1 from human erythrocytes or Hup1 from the microalga Chlorella kessleri. Positive transformants exhibited glucose uptake and grew in the dark in the presence of glucose (Zaslayskaia et al. 2001). The exciting trophic conversion of an obligate photoautotrophic diatom is a critical first step for successful large-scale cultivation using microbial fermentation technology.

Algae are highly suited as bioreactors for the large-scale production of foreign proteins for several reasons. First, they are relatively easy to culture as they will grow in a laboratory setting, subsisting on an inexpensive medium of simple salts. Second, unlike many cell lines, algae can be grown in continuous culture. Third, the cost for production on this platform is very low. Besides the tremendous cost advantage, the generation of initial transformants to production volumes can occur within a short period of time. This system is also highly scalable in that transformed algal lines can be grown in few milliliters to 500,000 liters in a cost effective manner as their growth medium can be recycled. Furthermore, both the chloroplast and nuclear genome of algae can be genetically transformed, opening the possibility of expressing multiple recombinant products in a single organism. This eukaryotic system also offers the advantages of post-translational modifications of expressed protein products. The economics, ease of use and flexibility of this system make it highly desirable for the expression of complex recombinant products.

Furthermore, previous methods for sequentially processing algal biomass into multiple biofuels products are described in co-pending International Patent Application PCT/US09/61127 which is hereby incorporated by reference. The utilization of microalgae as an advanced energy feedstock has been studied extensively, with applications being developed for biodiesel, bioethanol, and biohydrogen gas production (See e.g., Pienkos and Darzins, Biofuels Bioprod. Bioref., 3, 431-440 (2009)). Whole algae or algal oil extracts can be converted to different fuel forms such as biogas, liquid and gas transportation fuel, kerosene, ethanol, aviation fuel, biohydrogen implementing processing technologies such as anaerobic digestion, pyrolysis, gasification, catalytic cracking, enzymatic or chemical transesterification. Algae have higher photon conversion efficiency and can synthesize and accumulate large quantities of neutral lipids (biodiesel) and carbohydrates (bioethanol) along with other valuable co-products (such as astaxanthin, omega 3 fatty acids etc) from abundant and inexpensive raw materials (sunlight, CO2, inorganic nutrients found in wastewater). They can be grown on saline/coastal seawater and on non-crop lands (desert, arid and semi-arid land), resources for which there are no competing demands (See e.g., Huntley and Redalje, Mitigat. Adapt. Strat. Global Change. 12: 573-608. (2007)). Algae can utilize growth nutrients such as nitrogen and phosphorous from a variety of waste water sources (agricultural run-off, concentrated animal feed operations, and industrial and municipal waste water) thus providing a sustainable bioremediation of these waste water for economic benefits (See e.g., Shilton et al Environmental Science & Technology 42(16): 5958-5962. (2008)). They can also couple CO₂-neutral fuel production with CO₂ sequestration from other power industries which in turn generates carbon credits (See e.g., Dismukes et al., Current Opinion in Biotechnology 19: 235-240 (2008)). Compared to other advanced feedstock based on cellulosic ethanol, algal genomics and basic research is more advanced and getting increased momentum. Investors have already shown particular interest towards algae-based biofuel (Cleantech Investment Monitor, 2008) and most recently, industry leaders such as Shell, Chevron, ExxonMobil, and British Petroleum have also invested substantial resources (˜$ 1 billion) in developing algal-based biofuels (See e.g., Mascarelli, Nature 461: 460-461 (2009)).

Although several algal biofuel based companies have boomed in the last few years, most firms are still entangled in the non-trivial technology hurdles needed for cost effective production and extraction of biofuel from algal biomass. Cost competitiveness of biofuel is a major determinant of its success. Significant improvement in the efficiency, cost ability and ability to convert algal growing, lipid extraction, biofuel production needs to be perfected in a cost effective way in order to produce commercial biofuel (See e.g., Pienkos and Darzins, 2009, incorporated by reference above). A defined set of technology breakthroughs will be required to develop for the optimum utilization of algal biomass for commercial production of biofuel. With the existing technologies still not in a position to commercially produce biofuel with a competant price compared to fossil fuel, one of the main challenges facing algal biofuel sector is to attain high productivity while reducing the capital and operating cost. If biofuel alone is the product, the profit or return on investment is comparatively low for algal biofuel based farming which is another constraint from an investment perspective in using algal biofuel as an energy feedstock. Another major concern in the large-scale production of biofuel from algae is the higher cost of production.

One unique aspect of algae compared to other advanced feedstocks is the spectrum of species available for amenability for biofuel production. Further, diverse variety of uses and applications can be found in products ranging from omega 3 fatty acids, energy sources (including biodiesel and bioethanol), fertilizers, plastics, recombinant proteins, and pigments (Pienkos and Darzins, 2009, incorporated by reference above). Historically, algae have been used for different purposes. Some of these applications and technologies have even evolved into matured industries such as production of astaxanthin from algae. The multi-product paradigm from algae becomes particularly important in the present scenario as make it a perfect candidate feedstock for biorefinery concept. As algae produce many valuable products, developing an integrated algal processing (IAP) for the sequential production of various products from the same algal biomass can substantially reduce the cost of production which in turn will make it a profitable enterprise. For example, as the first step in IAP concept, harvested algal mass can be used for recombinant protein production. In the second step the algal oil and algal meal can be processed. From the algal oil high value omega 3 fatty acids such as docasahexanoic acid and eicosapentanoic acid can be separately processed. The rest of the oil can be used for biofuel production. Significant amount of glycerin is the end by-product of biodiesel production and recent studies have shown that glycerin in turn can be effectively utilized to grow more algal mass for the production process. Alternatively glycerin can also be used as a raw material for the bioproduction of chemicals such as 1, 3 propanediol via microbial or algal enzymatic methods. As some of these co-products such as omega 3 fatty acids and recombinant proteins have high demand and market value, adopting this approach can be a viable option for algal biofuel sector to attain a financial sustainability.

Because algae are able to produce both a variety of recombinant proteins and can be processed to produce biofuels, according to some embodiments, an algal biomass that has been transformed to express cellulases and hemicellulases as described above may be further transformed and/or processed to obtain other products and by-products including, but not limited to biofuels and other recombinant proteins.

Turning now to FIG. 3, a box diagram of co-processing algal and cellulosic feedstocks for biofuels production is shown. As shown an algal biomass that has been transformed to express cellulose and hemicellulase is harvested. The algal biomass is separated from the culture water, which contains the cellulose and hemicellulase. The harvested alga biomass undergoes oil separation to produce algal meal and algal oil. The algal oil undergoes Omega 3 separation to produce Omega-3 fatty acids (such as DHA, EPA and AA) and Oil-Omega 3. The oil-Omega3 is then processed via base catalyzed transesterification to produce glycerin and biodiesel. Glycerin can be further processed via algal or microbial fermentation conversion to produce 1,3 propanediol. In a simultaneous production cycle, the culture water containing the cellulose and hemicellulase can be processed, for example via protein column enrichment, gel filtration, affinity purification, or the like, to purify the cellulase and hemicellulase. The residual culture water can then be recycled back into the system while the cellulose and hemicellulase enzymes can be stored for use in producing bioethanol from cellulosic feedstock. An exemplary cellulosic feedstock processing plan is also shown. In this case, cellulosic material is harvested, compacted and transported, if necessary, to a processing plant. The material is then pretreated and detoxified. Various pretreatment methods can be used which included mild physical and chemical (acid treatment) processes. Alternatively, AFEX process can also be used for pretreatment. Various detoxification techniques such as water washing, overliming, ion exchange absorption or biodetoxification can be employed in the process. Solids and liquids are then separated and the thick liquid slurry are subjected to enzymatic hydrolysis using cellulases and hemicellulases (some or all of which may have been derived from the algal feedstock process.) Fermentation of break down products of cellulose and hemicelluloses results in bioethanol and co-products.

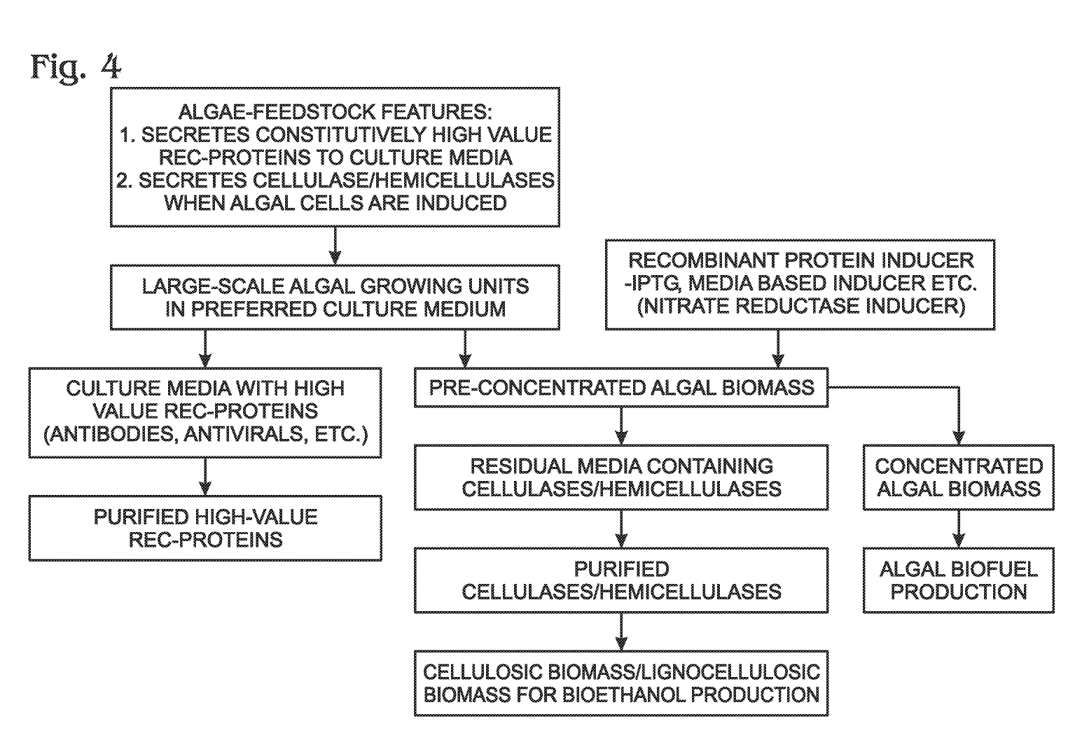

A method for co-producing cellulases, hemicellusase, biofuels and other recombinant proteins is shown in FIG. 4. In this embodiment, the alga feedstock is genetically engineered to consitutively secrete high value recombinant proteins (i.e. antibodies, antivirals, invertases, cytokines etc.) to the culture media and to only secrete cellulases/hemicellulase when the algal cells are induced. The transfored alga feedstock is then introduced to large-scale growing units including the desired culture medium where the algae is allowed to reproduce while secreting the desired recombinant protein into the culture media. If desired, the algae can be preconcentrated to reduce the culture media (for example to 1/100 volume) and obtain a live algal slurry. The slurry is then processed to obtain culture media containing the high value recombinant proteins and the pre-concentrated algal biomass. The recombinant proteins can be purified using known means including, but not limited to, column chromatography, gel filtration, affinity purification, etc. A recombinant protein inducer such as IPTG, media based inducer, nitrate reductase inducer or the like can then be introduced to the pre-concentrated algal biomass to induce secretion of cellulases and hemicellulases. The pre-concentrated and cellulase/hemicellulase producing algal mass can then be further concentrated (using, for example, a mechanical press or ultrafiltration) to produce a concentrated alga biomass and residual media containing cellulase/hemicellulase, both of which can be further processed, for example, as described with respect to FIG. 2.

Of course those of skill in the art will be appreciate that the algae could be transformed to constitutively express cellulase and/or hemicellulase and express other recombinant products (e.g. proteins, enzymes, etc.) when induced. Alternatively, all products (e.g. recombinant proteins, enzymes, etc.) could be constitutively expressed or only expressed when induced. As a further alternative some recombinant products could be expressed upon exposure to a first condition (temperature, chemical, pH level etc.), while other products could be expressed upon exposure to a second condition.

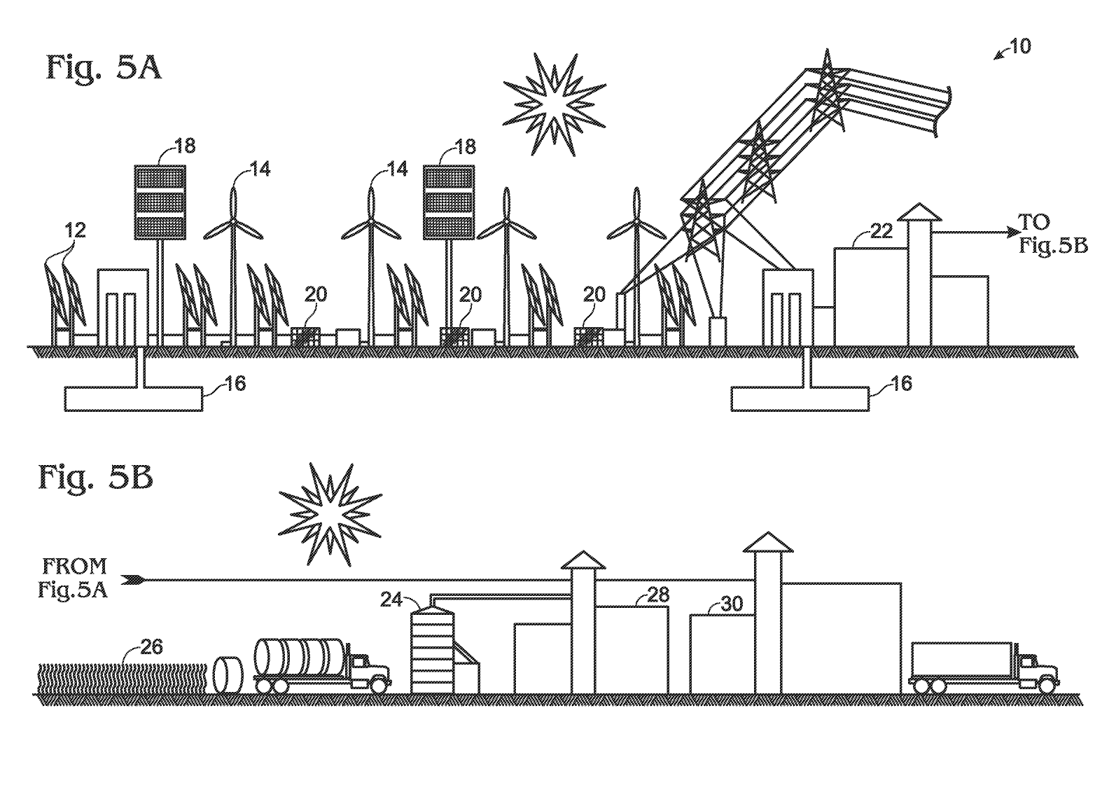

Turning now to FIG. 5, an exemplary integrated algal and cellulosic feedstock biorefinery is shown. In the exemplary embodiment, the biorefinery is shown to include a variety of renewable energy generators in order to create a biorefinery that is entirely independent of fossil fuels. It will be understood, however, that other traditional and alternative methods for producing energy could be incorporated into the biorefinery. In the exemplary biorefinery 10, energy is produced by soar energy grid 12, wind energy turbines 14, and geothermal energy collector 16. A carbon capture device 18 captures carbon dioxide for algal growth. The biorefinery includes arrays of algal greenhouses 20, which produce alga biomass, and an algal biorefinery 22, which may be configured to sequentially process co-products, biofuels, omega 3 fatty acids, recombinant proteins, algal meal, bulk chemicals etc. The biorefinery also includes a lignocelluloses hydrolysis plant 24, which hydrolyses the cellulose and hemi-cellulose in lignocelluloses (harvested from, for example, switch grass fields 26) using cellulase and hemicellulase produced in the algal biorefinery 22. Lignocellulose biorefinery 28 may then further process the switch grass, forest residues, agricultural crop residues or other sources of lignocelluloses to produce biofuels and other products. If desired, the biorefinery may include a fuel refinery 30 for final liquid fuel processing and storage.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications. The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise.

What is claimed is:

1. A method comprising:
transforming algae to express cellulase and/or hemicellulase;
allowing the algae to secrete the cellulase and/or hemicellulase into a culture medium; and
recovering the culture medium to obtain cellulase and/or hemicellulase.

2. The method of claim 1 further comprising delivering the cellulase and/or hemicellulase to cellulosic biomass in order to produce biofuels from the cellulosic biomass.

3. The method of claim 1 further comprising transforming the algae to also express a second desired recombinant product.

4. The method of claim 3 wherein the second desired recombinant product is constitutively expressed.

5. The method of claim 4 wherein the cellulase and/or hemicellulase is only expressed upon exposure of the algae to a predetermined condition.

6. The method of claim 3 further comprising allowing the algae to secrete the second desired recombinant product into a second culture medium.

7. The method of claim 6 further comprising recovering the second culture medium to obtain the second desired recombinant product.

8. The method of claim 1 further comprising processing the algae to obtain a biofuel.

9. A cellulase or hemicellulase that is obtained from an algal biofuel feedstock that has been transformed to express the cellulase or hemicellulase.

10. A biofuel that is obtained from cellulosic biomass that has been hydrolyzed by a cellulase or hemicellulase derived from an algal biofuels feedstock that has been transformed to express the cellulase or hemicellulase.

11. A recombinant product other than cellulase or hemicellulase that is obtained from an algal biofuels feedstock that has been transformed to express both the recombinant product and the cellulase or hemicellulase.

12. A biofuel that is obtained from an algal biofuel feedstock that has been transformed to express the cellulase or hemicellulase.
